(19) United States
(12) Patent Application Publication
(10) Pub. No.: US 2023/0011874 A1
(43) Pub. Date: Jan. 12, 2023

(54) DEVICE COMBINING POSITIONING AND INJECTION SYSTEMS FOR INJECTION WITHIN A BODY CAVITY

(71) Applicant: GINGKUI MEDICAL DEVICE TECHNOLOGIES DEVELOPMENT CO., LTD., TAICHUNG CITY (TW)

(72) Inventors: CHENG-SHING CHEN, Taichung City (TW); WEI-HUNG CHEN, New Taipei City (TW); PANG-LI YANG, Hsinchu County (TW)

(21) Appl. No.: 17/372,501

(22) Filed: Jul. 11, 2021

(51) Int. Cl. A61M 5/42

(52) U.S. Cl. CPC A61M 5/427; A61B 1/307

(57) ABSTRACT

A device combining positioning and injection systems for injection within a body cavity, comprises: a tubular housing surrounding an accommodating space and having an opening hole fluidly connected to the accommodating space; a curved channel fluidly connected to the accommodating space and the opening hole; an injection needle disposed inside the accommodating space and the curved channel; the injection needle having a piercing portion penetrating through the opening hole and configured to extend outward from or retract inward into the opening hole; a plurality of ultrasonic transducers installed at the tubular housing and at two opposite sides of the opening hole; wherein at least one of the ultrasonic transducers being used to transmit a detection signal, and at least one of the ultrasonic transducers being used to receive the detection signal; a monitoring unit connected to the ultrasonic transducers via a transmission unit for information transmission therebetween.

### BACKGROUND OF THE INVENTION

### 1. Technical Field

The present invention is a device that combines positioning and injection systems for precise injection of fluids within a body cavity, and in particular, it is a combination device for injection with detection and positioning functions.

### 2. Description of Related Art

Most of known body cavity injection devices perform insertion and retrieval in a linear direction, which is the same direction as that used for injection. However, many body cavity injection devices are often used in narrow and winding body channels, such as urethra and vagina, etc. Accordingly, the known injection devices using linear insertion and retrieval without bending capability are unable to successfully achieve injection into tissues of the body channel walls.

In addition, known body cavity injection devices are not able to determine the specific location and depth of injection in a convenient way, and medical and nursing personnel operating the injection device must rely on personal experience and feel to perform the injection. An external imaging system can be used in conjunction with known injection devices; however, such a system is unable to accurately image tissue in the vicinity of the injection device and often cannot display images in real-time. In view of the above drawbacks of known injection devices, there is a need for a novel device for use within body cavities, which device combines positioning and injection functions and enables physicians to determine the injection position and depth accurately.

### BRIEF SUMMARY OF THE INVENTION

The present invention provides a device combining positioning and injection systems for injection within a body cavity, and its primary objectives are to enable injection into the walls of body cavities and to assist medical and nursing personnel to determine the injection location and depth.

Another objective of the present invention is to facilitate replacement of the body-cavity injection system, and it therefore can be configured and manufactured as a disposable device in order to prevent cross infection during multiple sequential treatment processes.

To achieve the aforementioned objectives, the device combining positioning and injection systems for injection within a body cavity, comprises:

A tubular housing surrounding an accommodating space. The tubular housing includes an opening hole, and the opening hole is fluidly connected to the accommodating space.

A curved channel is fluidly connected to the accommodating space and the opening hole.

An injection needle is made of a flexible material, and the injection needle is disposed inside the accommodating space and the curved channel. The injection needle includes a piercing portion, and the piercing portion penetrates through the opening hole. In addition, the piercing portion is configured to extend outward from or retract inward into the opening hole.

A plurality of ultrasonic transducers are installed at the tubular housing, and the plurality of ultrasonic transducers are installed at two opposite sides of the opening hole. In addition, at least one of the ultrasonic transducers is used to transmit a detection signal, and another at least one of the ultrasonic transducers is used to receive the detection signal.

A monitoring unit is connected to the plurality of ultrasonic transducers via a transmission unit for information transmission therebetween.

From the above content, it can be understood that the tubular housing of the present invention includes the curved channel, and the curved channel may be utilized to allow the injection needle to bend and to extend outward from the opening hole, and the bending may reach 30 to 120 degrees, thereby overcoming the drawback of mere linear insertion and retrieval of the injection needle.

In addition, the ultrasonic transducers are disposed respectively on two opposite sides of the opening hole of the tubular housing of the present invention, which ultrasonic transducers uses well-known ultrasonic generation, transmission, detection and signal analysis methods to indicate both the depth and density of the body tissue near the transducers. Signals from the transducers are transmitted to the monitoring unit, which analyzes the signals to provide information on the contents of the volume of space near to the transducers, which space may include body tissue and the injection needle. This information can be presented on a visual display that allows the physician to understand the tissue density profile (i.e. tissue density as a function of the tissue’s distance from the transducer) and the piercing depth of the injection needle.

Furthermore, the curved channel is disposed inside the tubular housing, the injection needle is arranged inside the curved channel, and the ultrasonic transducers are also installed on the tubular housing. Accordingly, the combination device for positioning and injection within a body cavity may be modularized, thereby allowing the user to use such device as a disposable, discarding it after a single use.

### BRIEF DESCRIPTION OF THE SEVERAL VIEWS OF THE DRAWINGS

FIG. 1A is a schematic view of the present invention;

FIG. 1B is a partial schematic view of the present invention;

FIG. 2 is a schematic view showing the actuation of the present invention;

FIG. 3 is a rear view of the present invention;

FIG. 4 is a schematic view showing the image displayed by the monitoring unit of the present invention;

FIG. 5 is a schematic view showing the injection device of the present invention making an injection into the tissue; and

FIG. 6 is a schematic view showing the present invention combination device for positioning and injection within a body cavity being inserted into a cystoscope.

### DETAILED DESCRIPTION OF THE INVENTION

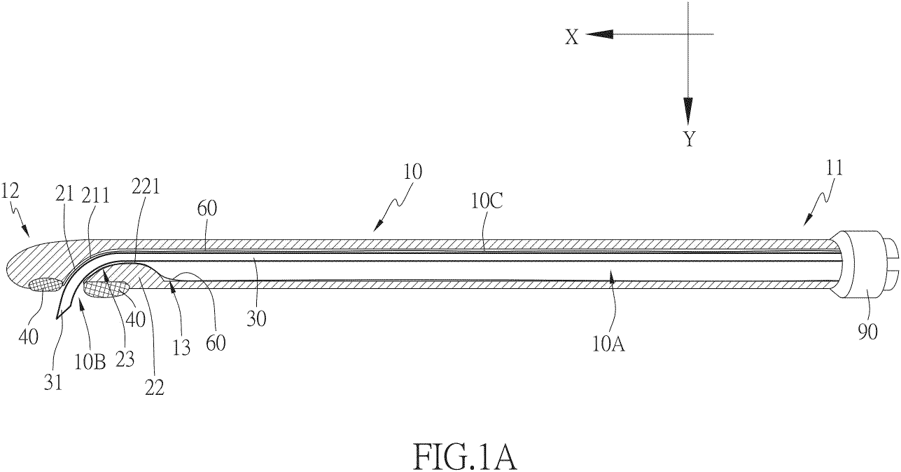

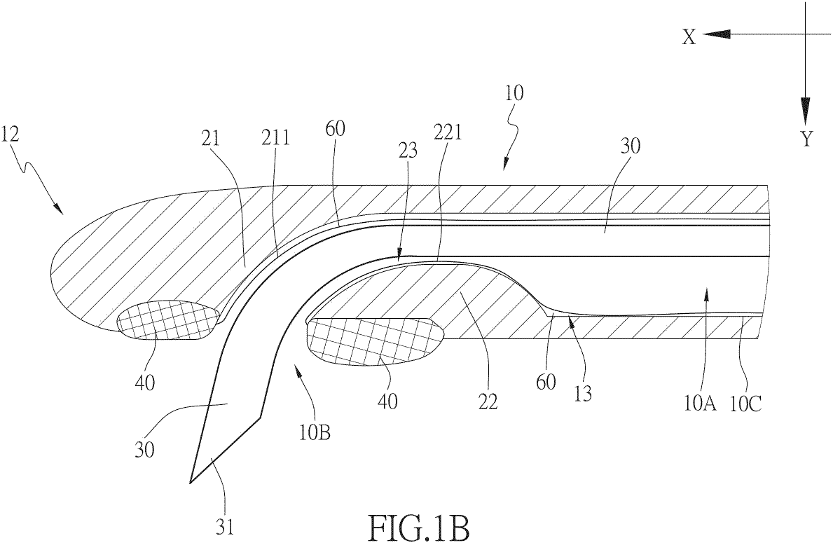

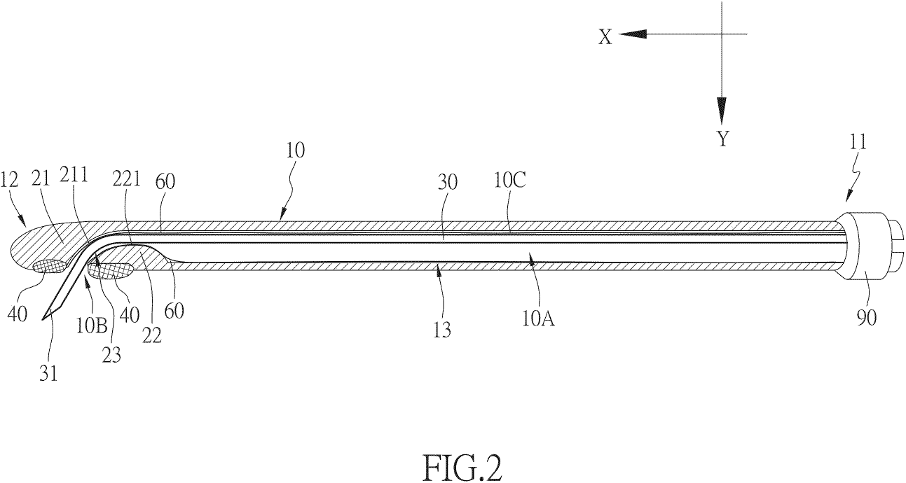

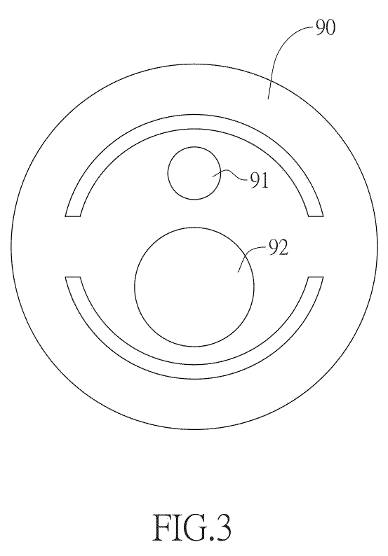

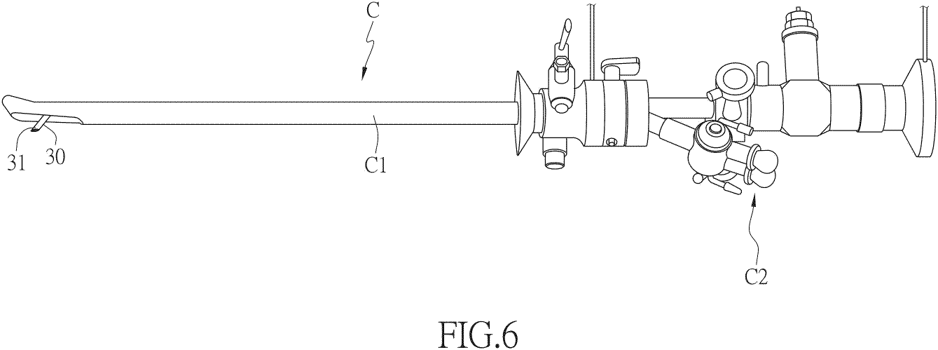

The present invention provides a device combining positioning and injection systems for injection within a body cavity for injection of fluids and detection of tissue. As shown in FIG. 1A to FIG. 6, the device is provided for installation inside a working channel of a sheath C1 of a known cystoscope C. The device combining positioning and injection systems for injection within a body cavity, comprises:

A tubular housing 10 has a circular column shape, and the tubular housing 10 extends along an axial direction X. The tubular housing 10 surrounds an accommodating space 10A, and the accommodating space extends along an axial direction X. The tubular housing 10 includes a first end 11 and a second end 12. The first end 11 is connected to a connector 90, and the connector 90 includes a first through hole 91 and a second through hole 92 formed thereon. The first through hole 91 and the second through hole 92 are fluidly connected to the accommodating space 10A. The connector 90 is provided for connecting to the cystoscope C. In addition, the second end 12 includes an opening hole 10B formed thereon. The opening hole 10B is located on the side of the tubular housing 10. The opening hole 10B is fluidly connected to the accommodating space 10A. The tubular housing 10 includes an inner side surface 10C, and the inner side surface 10C faces toward the accommodating space 10A.

In a preferred embodiment, the tubular housing 10 may be made of thermoplastic polyurethane (TPU), polyethylene terephthalate glycol (PETG) or polyethylene terephthalate (PET).

An abutment portion 21 is arranged inside the tubular housing 10 and is located at the second end 12. The abutment portion 21 includes a first curved side surface 211, and the first curved side surface 211 is connected to the inner side surface 10C.

A protrusion 22 is arranged inside the tubular housing 10, and the location of the protrusion 22 is adjacent to the abutment portion 21. The protrusion 22 protrudes outward from the inner side surface 10C, and the protrusion 22 is adjacent to the opening hole 10B. The protrusion 22 includes a second curved surface 221, and the second curved surface 221 faces toward the first curved surface 211. A curved channel 23 is formed between the first curved side surface 211 and the second curved side surface 221. The curved channel 23 is fluidly connected to the accommodating space 10A and the opening hole 10B. In this embodiment, the tubular housing 10 defines a radial direction Y, and the curved channel 23 includes a component extending along the radial direction Y indicated in FIG. 1B.

An injection needle 30 may be made of a flexible material and the injection needle 30 is disposed inside the accommodating space 10A and the curved channel 23. The injection needle 30 bends along the shape of the curved channel 23. The injection needle 30 has one end formed as a piercing portion 31. The other end of the injection needle penetrates the first through hole 91 and extends outward through an adapter C2 of the cystoscope C in order to allow a user to actuate and move its position, thereby allowing the piercing portion 31 to extend outward from the opening hole 10B to make the injection into the desired tissue or retract back into the opening hole 10B. In one embodiment, the injection needle 30 may be made of a metal, a nickel-titanium alloy, a titanium alloy, Teflon or a stainless steel; however, the present invention is not limited to such materials.

A plurality of ultrasonic transducers 40 are installed at the tubular housing 10. The plurality of ultrasonic transducers 40 are installed at opposite sides of the opening hole 10B; wherein at least one of the ultrasonic transducers 40 may be used to transmit a detection signal, and at least one of the ultrasonic transducers 40 may be used to receive the detection signal. The detection signal is a sonic wave, preferably between 1 and 10 mega Hz. In this embodiment.

The number of ultrasonic transducers 40 is two. The two ultrasonic transducers 40 are installed at opposite sides of the opening hole 10B. The two ultrasonic transducers 40 are located at different locations of the axial direction X.

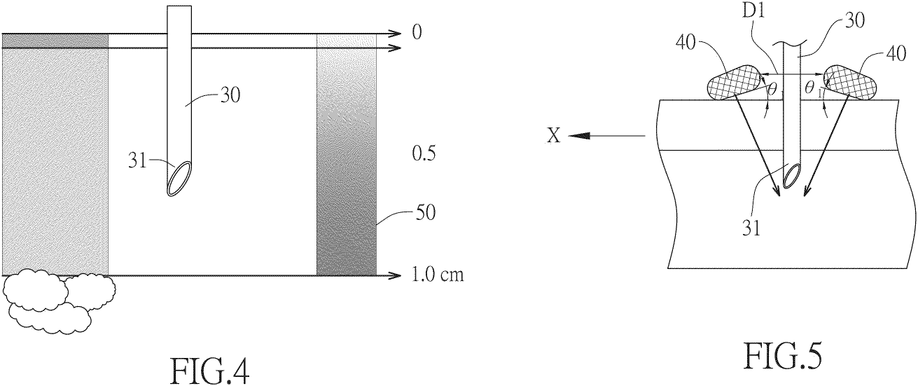

In a preferred embodiment, as shown in FIG. 5, a distance between the two ultrasonic transducers 40 refers to a first distance D1, and the first distance D1 is between 0.5~5 mm.

In a preferred embodiment, a detectable depth of tissue that that can be detected by the plurality of ultrasonic transducers 40 is between 0~2 cm, and 0 cm refers to the tissue surface of the detection.

In a preferred embodiment, the axial direction X defines an axial plane. A first angle θ1 is formed between the plurality of ultrasonic transducers 40 and the axial plane A, and the first angle θ1 is between 1 and 90 degrees

A monitoring unit 50 is connected to the plurality of ultrasonic transducers 40 via a transmission unit 60 for information and electrical power transmission therebetween. The tubular housing 10 includes an inner side surface 13, and the inner side surface 13 faces toward the accommodating space 10A. The transmission unit 60 is fixedly installed at the inner side surface 13 and extends along the tubular housing 10. The transmission unit 60 can comprise a transmission cable, printed circuit board, etc. Connection to the transmission unit 60 penetrates into the second through hole 92 and extends outward from the adapter C2 in order to connect to the monitoring unit 50 for information transmission therebetween. The monitoring unit 50 may display a screen showing information on the piercing operation of the injection needle 30.

In a preferred embodiment, the monitoring unit 50 contains a visual display indicating at least one of information includes density of tissue near the ultrasonic transducers, density profile of tissue near the ultrasonic transducers, location of the injection needle, or location of the piercing portion of the injection needle.

The above provides description of the structural configurations of main components of exemplary embodiments of the present invention. The actuation method and technical effects of the present invention are further explained in the following:

First, the device combining positioning and injection systems for injection within a body cavity is installed inside the working channel of the cystoscope C. Next, during the process when the medical practitioner inserts the cystoscope C into the body cavity, the medical practitioner is able to use the cystoscope’s optical system to observe and determine the lesion location for treatment and maneuver the cystoscope so that the ultrasonic transducers 40 and injection needle 30 arrive at the lesion location.

The medical practitioner then drives the injection needle 30 to move along the accommodating space 10A of the tubular housing 10. The method for driving the injection needle 30 can be achieved via direct pushing or a rotational method. In addition, when the injection needle 30 is driven by the user to move, the piercing portion 31 of the injection needle 30 is able to change its direction along with the curved channel 23. The curve angle is between 30 and 120 degrees, such that the piercing portion 31 is able to extend outward from the opening hole 10B at the lateral side of the tubular housing 10, thereby piercing into the human body cavity tissue adjacent to the tubular housing 10.

Furthermore, since the plurality of ultrasonic transducers 40 are located at opposite sides of the opening hole 10B, when the piercing portion 31 penetrates into the human body mucosa and muscle, at least one of the ultrasonic transducers 40 is able to send out a detection signal to the subcutaneous tissue in order to detect the location of the piercing portion 31. In addition, at least one of the ultrasonic transducers 40 is able to receive the detection signal and the transmission unit 60 conveys the necessary transducer signals to the monitoring unit 50, which may provide a visual display that enables the medical practitioner to clearly and readily understand the current piercing depth. Consequently, the invention enables the medical practitioner to make an injection of fluid precisely into the intended tissue location, to evaluate the success of the injection procedure and to pre-determine subsequent medical actions.

From the above description, it can be understood that the inside of the tubular housing 10 of the present invention includes the curved channel 23, and the curved channel 23 may be utilized to allow the injection needle 30 to bend and to extend outward from the opening hole 10B, and the bending may reach 30 to 120 degrees, thereby overcoming the drawback of mere linear insertion and retrieval of the injection needle 30.

In addition, the two opposite sides of the opening hole 10B of the tubular housing 10 of the present invention include the plurality of ultrasonic transducers 40, such that the ultrasonic transducers 40 can utilize the detection signal to penetrate through the mucous and muscle of human body and to identify the tissue density and depth to allow the medical practitioner to be able to understand the piercing depth of the injection needle 30 on the monitoring unit 50.

Furthermore, the curved channel 23 is disposed inside the tubular housing 10, and the injection needle 30 is arranged inside the curved channel 23, and the ultrasonic transducer 40 is also installed on the tubular housing 10. Accordingly, the combination device of positioning and injection systems within a body cavity may be modularized, thereby allowing the user to use such device or elements thereof as disposables for one-time use only.

What is claimed is:

1. A device combining positioning and injection systems for injection within a body cavity, comprising: a tubular housing surrounding an accommodating space; the tubular housing having an opening hole, and the opening hole fluidly connected to the accommodating space; a curved channel fluidly connected to the accommodating space and the opening hole; an injection needle made of a flexible material; the injection needle disposed inside the accommodating space and the curved channel; the injection needle having a piercing portion; the piercing portion penetrating through the opening hole, and the piercing portion configured to extend outward from or retract inward into the opening hole; a plurality of ultrasonic transducers installed at the tubular housing; the plurality of ultrasonic transducers installed at two opposite sides of the opening hole; wherein at least one of the ultrasonic transducers being used to transmit a detection signal, and at least one of the ultrasonic transducers being used to receive the detection signal; a transmission unit for communication between the plurality of ultrasonic transducers and a monitoring unit; and the monitoring unit that communicates with the plurality of ultrasonic transducers and provides information on the tissue near the plurality of ultrasonic transducers .

2. The device combining positioning and injection systems for injection within a body cavity according to claim 1, wherein the number of ultrasonic transducers is two, a distance between the two ultrasonic transducers refers to a first distance, and the first distance is between 0.5 and 5 mm.

3. The device combining positioning and injection systems for injection within a body cavity according to claim 1, wherein the tissue depth that can be detected by the plurality of ultrasonic transducers is between 0 and 2 cm.

4. The device combining positioning and injection systems for injection within a body cavity according to claim 1, wherein the tubular housing defines a radial direction, and the curved channel includes a component extending along the radial direction.

5. The device combining positioning and injection systems for injection within a body cavity according to claim 1, wherein the tubular housing includes a first end and a second end, and the first end is connected to a connector; the connector is provided for connecting to a cystoscope; and the opening hole is located at the second end.

6. The device combining positioning and injection systems for injection within a body cavity according to claim 5, wherein the connector includes a first through hole; the accommodating space is fluidly connected to the first through hole; the injection needle penetrates through the first through hole.

7. The device combining positioning and injection systems for injection within a body cavity according to claim 1, wherein the material of the injection needle is a nickel-titanium alloy, a titanium alloy, Teflon or a stainless steel.

8. The device combining positioning and injection systems for injection within a body cavity according to claim 1, wherein the tubular housing includes an inner side surface; the inner side surface faces toward the accommodating space; the transmission unit is fixedly installed at the inner side surface and extends along the tubular housing.

9. The device combining positioning and injection systems for injection within a body cavity according to claim 1, wherein the tubular housing extends along an axial direction; the axial direction defines an axial plane; a first angle is formed between the plurality of ultrasonic transducer and the axial direction, and the first angle is between 1 and 90 degrees.

10. The device combining positioning and injection systems for injection within a body cavity according to claim 1, wherein the tubular housing is made of a material of thermoplastic polyurethane (TPU), polyethylene terephthalate glycol (PETG) or polyethylene terephthalate (PET).
